(19) United States
(12) Patent Application Publication
(10) Pub. No.: US 2017/0007817 A1
(43) Pub. Date: Jan. 12, 2017

(54) Intravenous Tube Cap

(71) Applicant: Daniel Storie, Syracuse, NY (US)

(72) Inventor: Daniel Storie, Syracuse, NY (US)

(21) Appl. No.: 14/793,207

(22) Filed: Jul. 7, 2015

(51) Int. Cl. A61M 39/20; A61M 5/14; A61M 5/162; A61M 5/32

(52) U.S. Cl. CPC A61M 39/20; A61M 5/3202; A61M 5/1414; A61M 5/162

(57) ABSTRACT

The present disclosure is directed to an IV cap that has a large grasping area and defines opening to allow the cap to be hung on an IV pole or other mount. The IV cap, once opened from a sterile packaging will connect to a female threaded, luer lock IV component: most likely a form of IV tubing or syringe. The IV cap will plug the connected IV tubing or syringe, maintaining sterility within the tubing system. The feature of the perforated tab of the IV cap will allow the user to hang the cap loop on an IV pole wire or hook.

### FIELD OF THE INVENTION

The present invention is directed generally to intravenous tubing caps, and, more particularly, to intravenous tubing caps that have a large grasping area and a slot for hanging the intravenous tubing cap on an IV pole or other hanging implement.

### BACKGROUND

Intravenous (“IV”) therapy offers the fastest route to introduce fluid and medication into the body and thus has achieved near ubiquitous status in hospitals worldwide. All current IV systems employ the use of IV tubes which carry the medicine or fluid to a hypodermic needle or other implement for administering the medicine or fluid directly into the vein of a patient. To keep the IV tubes sterile between uses, current guidelines suggest placing a sterile cap in the end of the IV tube which protects the tip of the IV tube from potential contaminants.

However, IV caps currently in circulation around hospitals and health clinics carry a significant risk of contamination in the course of ordinary use. Existing IV caps are small in size with minimal surface to grasp the product during connection to a syringe or IV tubing. The small grasping area on the caps creates a risk the user will touch the sterile connection threads thus potentially contaminating the IV cap and/or tubing. Additionally, once the cap is connected, there is no means to hang the tubing to prevent it from coming in contact with the floor. If the user attempts to lay the tubing over the infusion pump it will most likely fall to the floor if the IV pole is moved. Once on the ground, the tubing will likely be stepped on or run over by medical equipment that can result in contamination and/or perforation of the tubing. Although invisible to the naked eye, bacteria and microbes on the contaminated tubing will most likely be touched when the user attempts to disconnect or reconnect the IV tubing at a later time.

Accordingly, there exists a need in the art for an IV cap that has a large grasping area to mitigate the risk that a user may touch the sterile plug, and that has an opening to allow the cap to be hung on an IV pole or other mount.

### SUMMARY OF THE INVENTION

The present disclosure is directed to an IV cap that has a large grasping area and an opening to allow the cap to be hung on an IV pole or other mount. The IV cap, once opened from a sterile packaging will connect to a female threaded, luer lock IV component: most likely a form of IV tubing or syringe. The IV cap will plug the connected IV tubing or syringe, maintaining sterility within the tubing system. The feature of the perforated tab of the IV cap will allow the user to hang the cap loop on an IV pole wire or hook.

The ultimate purpose for the looped IV cap is to help reduce the risk of hospital-acquired infections related to central or peripheral IV line contamination. The long, flat tab of the IV cap distal end to the male connection end creates a surface to grasp the cap during the connection process. This will maintain an appropriate distance between the sterile cap threads and the gloved or ungloved fingers of the user. In addition, once the cap is secured, the hole of the IV cap can be looped onto any IV pole hanging-wire or hook. Securing the IV tubing by the loop will prevent the tubing from ever coming in contact with ground or sterile field, especially during movement of the IV pole.

### BRIEF DESCRIPTION OF THE DRAWINGS

The various embodiments of the invention will be better understood when read in conjunction with the following drawings:

FIG. 1A shows a front view of an IV tube cap according to an embodiment;

FIG. 1B shows a side view of an IV tube cap according to an embodiment;

FIG. 2A shows a front view of an IV tube cap according to an embodiment;

FIG. 2B shows a side view of an IV tube cap according to an embodiment;

FIG. 3 shows a side view of an IV tube cap in use according to an embodiment;

FIG. 4A shows a front view of an IV tube cap according to an embodiment; and

FIG. 4B shows a front view of an IV tube cap according to an embodiment.

### DETAILED DESCRIPTION OF THE EMBODIMENTS

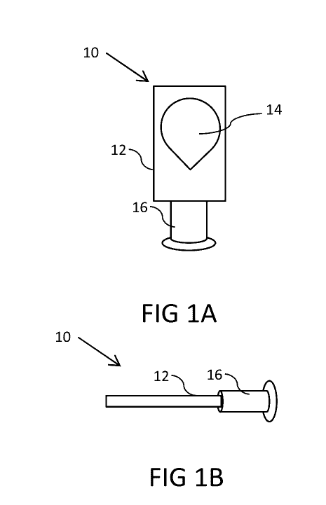

Referring now to the drawings wherein like reference numerals refer to like parts throughout, there is seen in FIG. 1A, a IV tube cap 10 according to an embodiment. As shown, IV tube cab 10 comprises a plug 16 that is dimensioned to insert into and seal an IV tube (not shown), syringe, or any other medical device having a female-threaded, luer lock IV component. IV tube cap 10 further comprises a tab 12 that is dimensioned to permit a user to easily hold and use the IV tube cap 10 without touching the plug 16. Tab 12 may be perforated, or otherwise formed to define a hanging opening 14. Hanging opening 14 is dimensioned to fit over a portion of an IV pole so that a user to may hang IV tube cap 10, and an IV tube engaged with IV tube cap 10, on an IV pole. Thus, the IV tube, when not in use, will be sealed with a sterile, untouched plug, and hung on an IV pole where it will not fall or otherwise be damaged or exposed to contaminants. While hanging opening 14 is shown to as a teardrop shape, a person of ordinary skill in the art will appreciate that opening 14 may be any shape (i.e., circular, oblong, triangular) that is suitable for hanging tab 12 on an IV pole.

FIG. 1B shows an alternate view of IV tube cap 10. As shown, tab 12 may be narrower than the diameter of plug 16, however one of ordinary skill in the art will appreciate that tab 12 may instead be thicker than the diameter of plug 16, or alternately, tab 12 may vary in thickness.

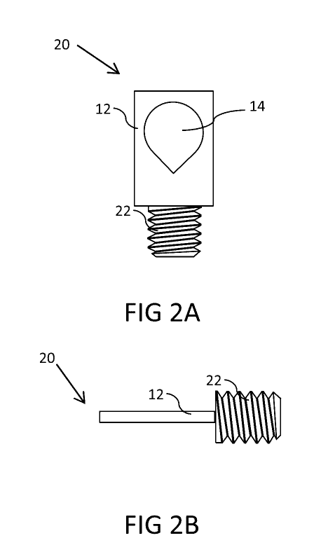

FIG. 2A shows an alternate embodiment of IV tube cap 10, shown here as IV tube cap 20. IV tube cap 20 has a fully threaded plug 22 which may be engaged with IV tubes having a fully threaded female end, as will be appreciated by a person of ordinary skill in the art. One of ordinary skill will also appreciate that plug 16, 22, may take on a variety of shapes and configurations. Plug 16, 22 may be fully threaded or partially threaded. In an alternate embodiment, plug 16, 22 may not be threaded at all, but may be dimensioned to tightly insert into an IV tube and form a seal therein. Alternately, plug 16, 22 may be have a sealing ring, such as an O-ring, to form a seal. FIG. 2B similarly shows a side view of IV tube cap 10 with fully threaded plug 22.

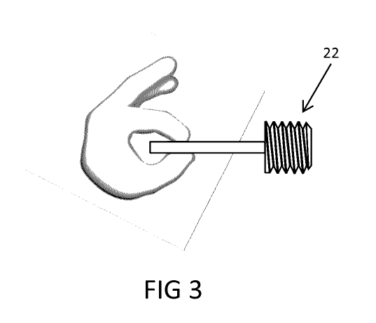

FIG. 3 depicts how tab 12 may be grasped by a user. As shown, the elongated tab 12 allows the user to grasp the IV tube cap 10 without risking contact with sterile plug 22 (or plug 16).

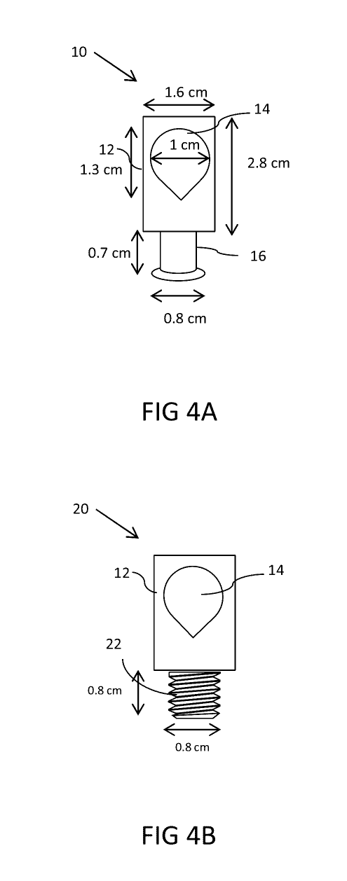

FIG. 4A shows an embodiment of plug 10 with exemplary dimensions. As shown, tab 12 may be 2.8 cm long and 1.6 cm wide. One of ordinary skill will recognize that tab 12 and plug 16 may have any number of dimensions and still fall within the scope and spirit of this disclosure. In particular, tab 12 may be any length sufficient to easily grasp tab 12 without touching plug 16, 22. With this in mind, in an exemplary embodiment, tab 12 may be any length greater than 2 cm long. For ease of packing and handling, it may be advantageous to limit the length of the tab to 6 cm, however, some embodiments may extend beyond that length.

Similarly, hanging opening may be 1.3 cm long and 1 cm wide. In an alternate embodiment, hanging opening may be any dimension less than 1.5 cm long and 1 cm wide. One of ordinary skill in the art will appreciate that opening 14 may be any dimension sufficient to hang IV tube cap 10 or 20 on an IV pole or other hanging implement, such as a hook.

As shown in FIG. 4A plug 16 may be 0.7 cm long, and have a diameter of 0.8 cm. As shown in FIG. B, plug 22 may be, in an exemplary embodiment, 0.8 centimeters long and 0.8 centimeters wide. One of ordinary skill in the art will recognize that the dimensions of plug 16, 22 will depend upon the dimensions of IV tube, syringe, or other device that will receive and be sealed by plug 16, 22. Therefore, plug 16, 22 may be any dimensions sufficient to plug and seal an IV tube, syringe, or other device.

While various embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, embodiments may be practiced otherwise than as specifically described and claimed. Embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

The above-described embodiments of the described subject matter can be implemented in any of numerous ways. For example, some embodiments may be implemented using hardware, software or a combination thereof. When any aspect of an embodiment is implemented at least in part in software, the software code can be executed on any suitable processor or collection of processors, whether provided in a single device or computer or distributed among multiple devices/computers.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

What is claimed is:

1. An IV tube cap, comprising:
a plug dimensioned to insert and seal an IV component;
a tab connected to the plug, and dimensioned to allow a user to grasp the tube cap without touching the plug; wherein the tab defines an opening which is dimensioned to receive a hanging implement.

2. The IV tube cap of claim 1, wherein the IV component is an IV tube.

3. The IV tube cap of claim 1, wherein the IV component is a syringe.

4. The IV tube cap wherein the plug is threaded to engage a female leur lock IV component.

5. The IV tube cap of claim 4, wherein the plug is partially threaded.

6. The IV tube cap of claim 4, wherein the plug is fully threaded.

7. The IV tube cap of claim 1, wherein the tab is greater than 2 centimeters long.

8. The IV tube cap of claim 7, wherein the tab less than 6 centimeters long.

9. The IV tube cap of claim 1, wherein the opening is less than 1.5 centimeters long.

10. The IV tube cap of claim 1, wherein the opening is less than 1 centimeters wide.
